(19) United States
(12) Patent Application Publication
(10) Pub. No.: US 2026/0241095 A1
(43) Pub. Date: Aug. 20, 2026

(54) NEEDLE RETRACTION ASSEMBLY FOR A DEVICE FOR DELIVERING INSULIN

(71) Applicant: AITA BIO INC., New York, NY (US)

(72) Inventor: Russell Cole, River Vale, NJ (US)

(21) Appl. No.: 19/156,293

(22) PCT Filed: Feb. 15, 2024
(86) PCT No.: PCT/US24/15955

§ 371 Date: Aug. 13, 2025

Related U.S. Application Data
(60) Provisional application No. 63/446,653, filed on Feb. 17, 2023.

(51) Int. Cl. A61M 5/142

(52) U.S. Cl. CPC A61M 5/14248; A61M 2005/14256; A61M 2202/0007; A61M 2202/04; A61M 2205/3303; A61M 2205/3327; A61M 2210/04; A61M 2230/201

(57) ABSTRACT

An infusion system comprising: a device configured to deliver medication to a user through an infusion catheter, wherein the device includes a housing configured to receive the infusion catheter and to maintain the infusion catheter in the user after insertion; and a detachable activation mechanism for deploying the device for delivering medication to the user, wherein the detachable activation mechanism comprises a trigger and a retraction mechanism that includes an introducer needle for introducing the infusion catheter into the user, wherein the detachable activation mechanism is configured to releasably connect to the housing, wherein the detachable activation mechanism is configured to insert the infusion catheter into the user, automatically release the detachable activation mechanism from the housing responsive to activation of the trigger and automatically retract the introducer needle into the needle retraction assembly to shield the needle upon the automatic release of the detachable activation mechanism.

### CROSS REFERENCE TO RELATED APPLICATIONS

This application claims priority to U.S. provisional application No. 63/446,653, filed Feb. 17, 2023, entitled “Needle Retraction Assembly For A Device For Delivering Insulin,” which is incorporated by reference herein.

### FIELD OF THE INVENTION

The present invention relates to a needle retraction assembly for a device for delivering insulin or other medication.

### BACKGROUND OF THE INVENTION

Insulin pumps help people with diabetes to conveniently manage their blood sugar. These devices deliver insulin at specific times. Insulin patch pumps or pods are one type of insulin pump. The pods are wearable devices that adhere to the skin of a user using an adhesive patch. The pods deliver insulin from a chamber and internal cannula based on separately acquired CGM sensor readings. The pods are controlled wirelessly with a handheld controller.

It would be advantageous to provide improvements to insulin pumps described above.

### SUMMARY OF THE INVENTION

A needle retraction assembly for a device for delivering insulin or other medication is disclosed.

In accordance with an embodiment of the present disclosure, an infusion system comprising: a device configured to deliver medication to a user through an infusion catheter, wherein the device includes a housing configured to receive the infusion catheter and to maintain the infusion catheter in the user after insertion; and a detachable activation mechanism for deploying the device for delivering medication to the user, wherein the detachable activation mechanism comprises a trigger and a retraction mechanism that includes an introducer needle for introducing the infusion catheter into the user, wherein the detachable activation mechanism is configured to releasably connect to the housing, wherein the detachable activation mechanism is configured to insert the infusion catheter into the user, automatically release the detachable activation mechanism from the housing responsive to activation of the trigger and automatically retract the introducer needle into the needle retraction assembly to shield the needle upon the automatic release of the detachable activation mechanism.

In accordance with another embodiment of the disclosure, a system for inserting an infusion catheter into a user, comprising: a device configured to deliver medication to a user through an infusion catheter, wherein the device includes a housing configured to receive the infusion catheter and to maintain the infusion catheter in the user after insertion; the detachable activation mechanism for deploying the device on the user for delivering medication to the user, wherein the detachable activation mechanism is configured to releasably connect to the housing, wherein the detachable activation mechanism comprises a trigger and wherein the detachable activation mechanism is configured to insert the infusion catheter into the user and to automatically release the detachable activation mechanism from the housing responsive to activation of the trigger.

In accordance with another embodiment of the disclosure, an infusion system comprising: a detachable activation mechanism for deploying a device on the user for delivering medication to the user through an infusion catheter and detaching from the device subsequent to deployment of the device, wherein the detachable activation mechanism includes a needle retraction assembly comprising an introducer needle configured to introduce the infusion catheter into the user, wherein needle retraction assembly is configured to move the introducer needle from (a) a first position wherein the introducer needle is not inserted in the user, to (b) a second position wherein the introducer needle is inserted into the user and the catheter is introduced into the user, and then to (c) a third position wherein the introducer needle is in a retracted position within the needle retraction assembly shielded from the user while the catheter is maintained in the user.

In accordance with yet another embodiment of the disclosure, an infusion system comprising: a device for delivering medication to a user; a cartridge assembly including an infusion catheter for infusing the medication into tissue of the user and an analyte sensor for measuring an analyte in the user, a detachable activation mechanism for deploying the device on the user for delivering medication to the user and for detaching from the device subsequent to deployment of the device, wherein the detachable activation mechanism comprises a needle retraction assembly mounted on the cartridge assembly, the needle retraction assembly includes a first introducer needle for introducing the infusion catheter into the user and a second introducer needle for introducing the analyte sensor in the user, wherein the detachable activation mechanism is configured to cause the cartridge assembly to move from (1) a first position wherein the first and second introducer needles are in a retracted position within the device for delivering medication to the user to (2) a second position within the device, wherein the first and second introducer needles are in a deployed position inserted into a subcutaneous tissue of the user's skin along with the infusion catheter and the analyte sensor, respectively.

In yet another embodiment of the disclosure, a detachable activation mechanism for deploying a device on the user for delivering medication to the user and detaching from the device subsequent to deployment of the device, wherein the device for delivering medication including an infusion catheter for infusing the medication into the user, the detachable activation mechanism comprising: a needle retraction assembly comprising an introducer needle configured to introduce the infusion catheter into the user, wherein needle retraction assembly is configured to move the introducer needle from (a) a first position wherein the introducer needle along with infusion catheter are not inserted in the user, to (b) a second position wherein the introducer needle is in a deployed position inserted into the user along with the catheter, and then to (c) a third position wherein the introducer needle is in a retracted position within the needle retraction assembly shielded from the user while the catheter is maintained in the user.

### BRIEF DESCRIPTION OF DRAWINGS

FIG. 1 depicts a perspective view of an example retraction assembly (also referred to as needle retraction assembly), mounted on a cartridge assembly before use, for a device for delivering insulin of an infusion system.

FIG. 2 depicts a cross sectional view of the needle retraction assembly and cartridge assembly of FIG. 1 after needle insertion, but before needle retraction is triggered.

FIG. 3 depicts a cross sectional view of the needle retraction assembly and cartridge assembly of FIG. 1, but after needle retraction.

FIG. 4 depicts an exploded view of the components of the needle retraction assembly in FIG. 1.

FIG. 5 depicts the cartridge assembly and the needle retraction assembly in FIG. 1 with an insulin catheter and CGM sensor.

FIG. 6 depicts a perspective view of a catheter and cartridge assembly in FIG. 1 fluidically and electrically connected to the device for delivering insulin with some of the insertion mechanism components hidden for visual clarity.

FIGS. 7 and 8 depict rear perspective views, respectively, of the device for delivering insulin of the infusion system along with the needle retraction assembly and the cartridge assembly in FIG. 1 in different activation configurations with some of the insertion mechanism components hidden for visual clarity.

FIG. 9 depicts a cross sectional view of the needle retraction assembly and the cartridge assembly in FIG. 1 in a pre-activation configuration.

FIG. 10 depicts a perspective view of the needle retraction assembly and the cartridge assembly shown in FIG. 9.

FIG. 11 depicts a cross sectional view of the needle retraction assembly and the cartridge assembly shown in FIG. 10.

FIGS. 12 and 13 depict cross sectional views of detachable activation mechanism of the infusion system, in part, showing the device for delivering insulin in a pre-activation state and an intermediary state during insertion, respectively.

FIG. 14 depicts a cross sectional view of the detachable activation mechanism of the infusion system, in part, showing the introducer needles in a deployed configuration.

FIGS. 15 and 16 depict a front perspective view and a cross sectional view thereof, respectively of the needle retraction assembly with the release lever rotated in the release position, but before the needles have retracted in FIG. 1.

FIG. 17 depicts a cross-sectional view of the infusion system in which the detachable activation mechanism is in a post activation configuration.

FIG. 18 depicts a bottom view detachable activation mechanism in FIG. 17.

FIG. 19 depicts the device for delivering insulin shown in FIGS. 6-8 in a wear configuration after the sensor and catheter have been inserted, the introducer needles have been retracted and the detachable activation mechanism removed.

FIG. 20 depicts a view of the components of the infusion system in FIGS. 12-14 in an exploded configuration.

FIG. 21 depicts an exploded view of the components of another example needle retraction assembly.

FIGS. 22-23 depict perspective views of an example release housing of the needle retraction assembly in FIG. 21 wherein a lever of the housing is shown in unflexed and flexed configurations respectively.

FIG. 24 depicts a perspective view of the example needle retraction assembly in FIG. 21, in assembled form, for a device for delivering insulin (of an infusion system) in a pre-activation configuration.

FIG. 25 depicts a cross sectional view of the example needle retraction assembly in FIG. 24.

FIG. 26 depicts a cross-sectional view of the example needle retraction assembly in FIG. 24, but after needle retraction.

FIG. 27 depicts an enlarged perspective view of the needle retraction assembly in FIG. 21, in assembled form, mounted on cartridge assembly.

FIG. 28 depicts a perspective view of the needle retraction assembly in FIG. 21 and cartridge assembly fluidically and electrically connected to the device for delivering insulin of the infusion system (before device for delivering insulin is assembled into a detachable activation mechanism).

FIG. 29 depicts a rear view of the needle retraction assembly and cartridge assembly in FIG. 28 with the insertion spring and rod.

FIG. 30 depicts a rear perspective view of the needle retraction assembly in FIG. 21 in assembled form with some of the insertion mechanism components hidden for clarity, and cartridge assembly after introducer needle insertion.

FIG. 31 depicts a rear perspective view of the needle retraction assembly mounted on the cartridge assembly in FIG. 29 (without device for delivering insulin) in which the introducer needles are exposed for insertion.

FIG. 32 depicts a front perspective view of the needle retraction assembly mounted on the cartridge assembly in FIG. 31.

FIG. 33 depicts a cross sectional view of the needle retraction assembly mounted on the cartridge assembly in FIG. 32.

FIG. 34 depicts a cross sectional view of the infusion system in which the device for delivering insulin is a pre-activation configuration.

FIG. 35 depicts a cross sectional view the infusion system in FIG. 34 in which the device for delivering is in an intermediary state wherein the cartridge assembly is in a partially advanced configuration.

FIG. 36 depicts an enlarged cross sectional view the infusion system in FIG. 35 wherein the cartridge assembly is in a fully advanced configuration and the introducer needles are fully inserted into the user.

FIG. 37 depicts a perspective view of the needle retraction assembly of FIG. 21 with a level catch disengaged.

FIG. 38 depicts a cross sectional view of the needle retraction assembly in FIG. 37.

FIG. 39 depicts a full cross-sectional view of the infusion system shown in FIG. 34 in which the detachable activation mechanism is in a post activation configuration.

FIG. 40 depicts a bottom view of the detachable activation mechanism in FIG. 39.

FIG. 41 depicts a perspective view of another example needle retraction assembly.

FIG. 42 depicts a block diagram of components of the example infusion systems described herein.

### DETAILED DESCRIPTION OF THE INVENTION

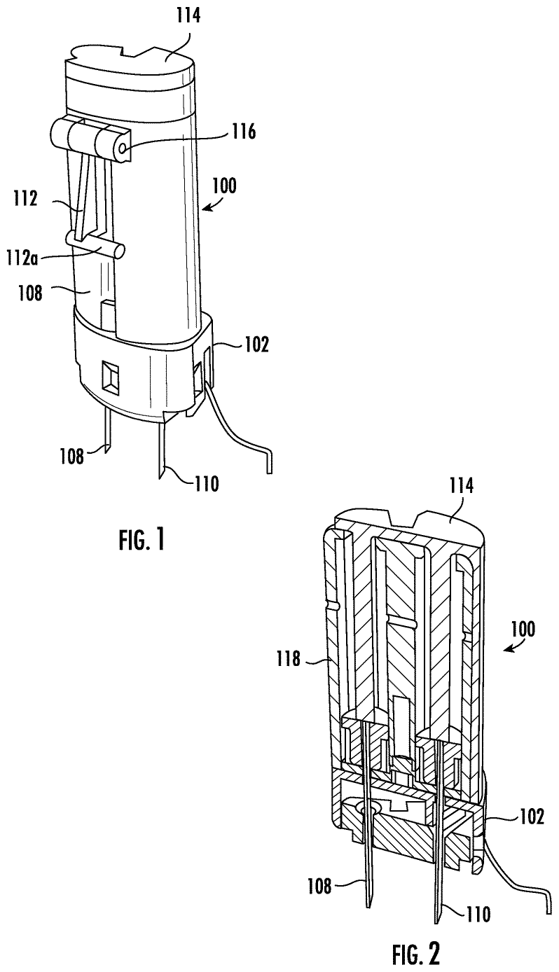

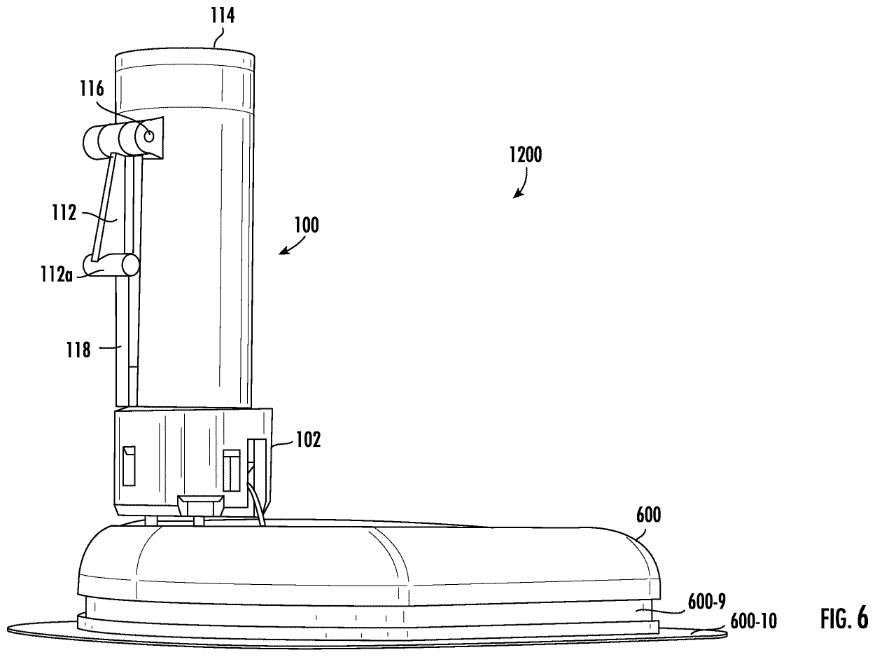

FIGS. 1-3 depict views of an example retraction assembly 100 (or needle retraction assembly 100) for device 600 for delivering insulin or other medication (shown in FIG. 6 for example). Retraction assembly 100 part of and housed inside detachable activation mechanism 1202. Detachable activation mechanism 1202 is part of infusion system 1200 along with device 600 for delivering insulin as described in detail below.

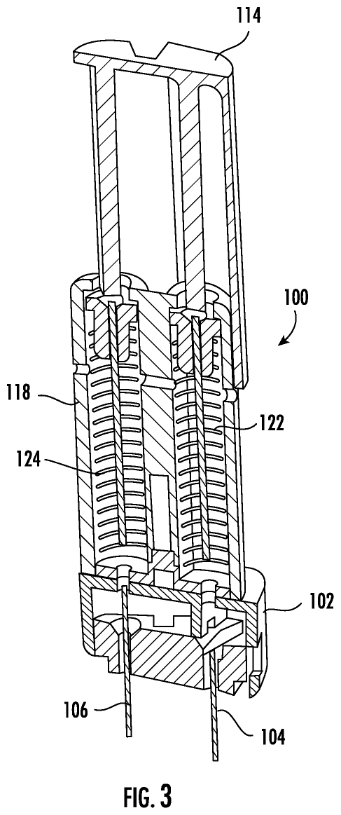

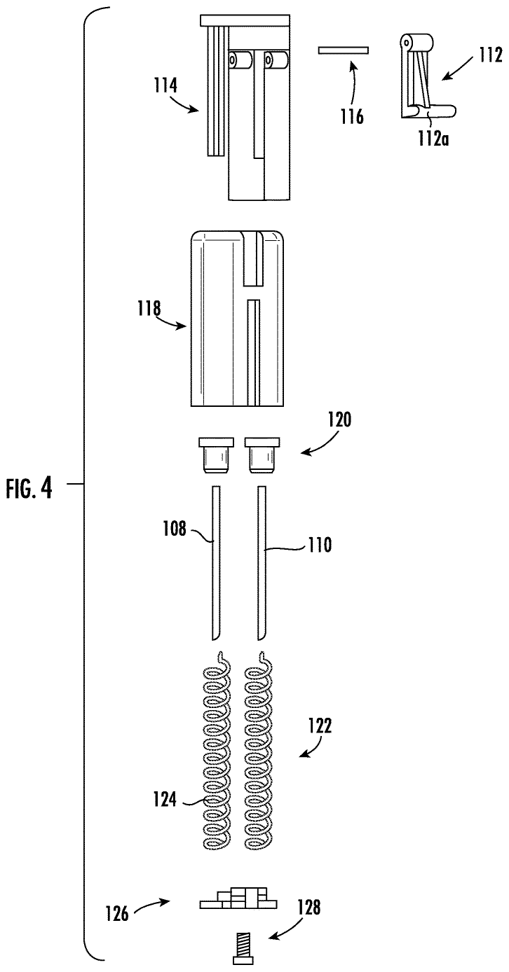

Specifically, FIG. 1 depicts a perspective view of example needle retraction assembly 100, mounted on a cartridge assembly 102 before use, for device 600 for delivering insulin of an infusion system. FIG. 2 depicts a cross sectional view of the needle retraction assembly 100 and cartridge assembly 102 of FIG. 1 after needle insertion, but before needle retraction is triggered. FIG. 3 depicts a cross sectional view of the needle retraction assembly 100 and cartridge assembly 102 of FIG. 1, but after needle retraction.

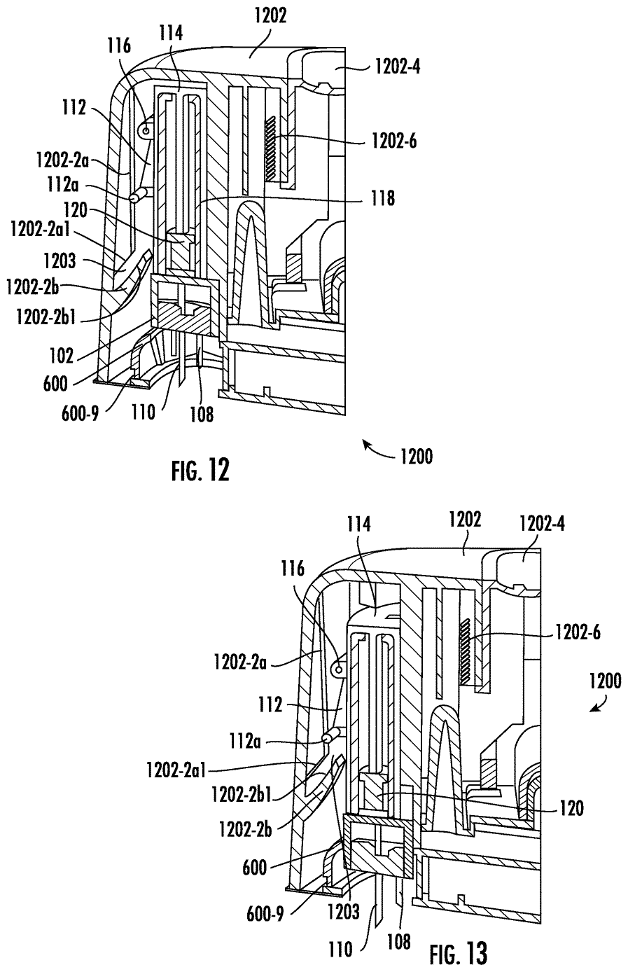

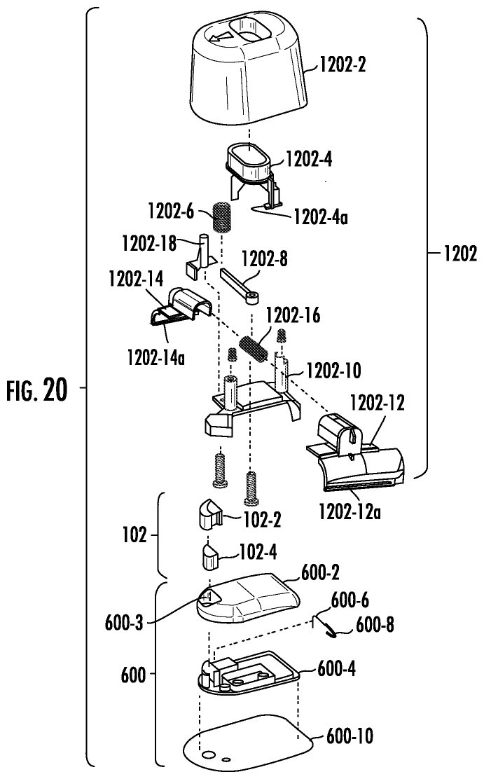

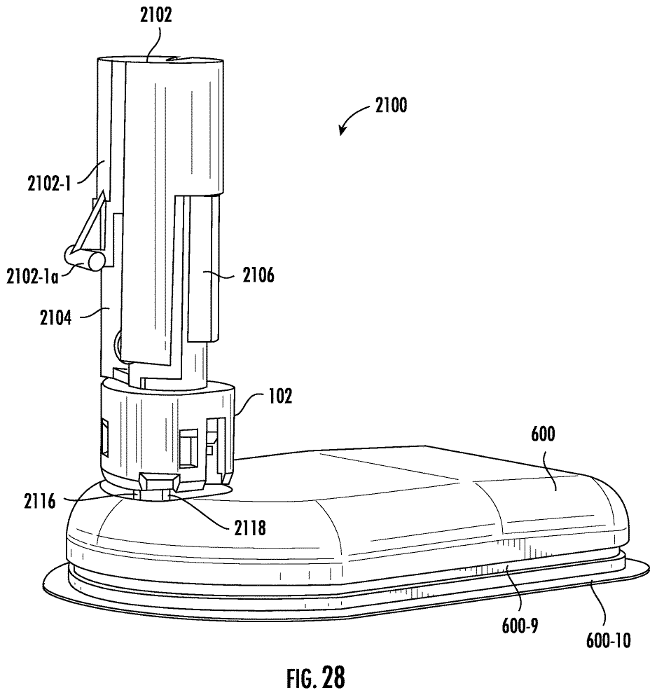

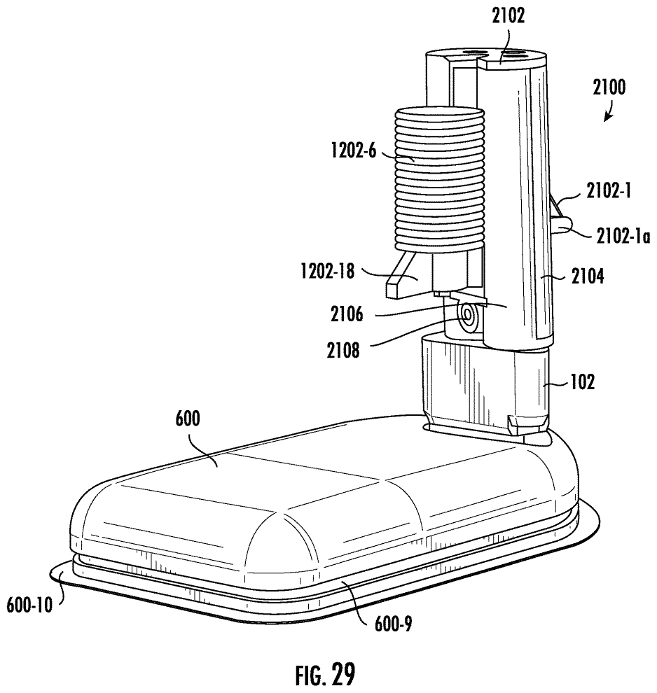

Needle retraction assembly 100 is within detachable activation mechanism 1202 and part of infusion system 1200 as shown in FIGS. 12 and 13 and discussed below. Needle retraction assembly 100 is assembled or mounted onto cartridge assembly 102 (described in detail below) of device 600 that incorporates an infusion catheter and continuous glucose monitoring (CGM) sensor to be advanced into the subcutaneous layer of the tissue as described in more detail below. Device 600 (also referred to as a delivery device 600 or pod) is a component of infusion system 1200 (as shown in FIGS. 12 and 13, exploded view in FIG. 20, FIGS. 28-30 for example) for infusing insulin or other fluid medication into a user (patient).

In brief, device 600 as described in more detail below comes preassembled for single use with detachable activation mechanism 1202. Upon activation, detachable activation mechanism 1202 releases a spring which drives dual introducer needles 108, 110 along with CGM sensor 106 and infusion catheter 104, respectively, into the skin. Introducer needles 108, 110 remain inserted and coupled to the catheter and sensor, respectively until the detachable activation mechanism 1202 separates from the device 600 whereby retraction assembly 100 is triggered resulting in the retraction of needles 108, 110 inside a housing. The needle tips are locked inside the retraction assembly housing so that pressing on the housing cannot result in the exposure of needles 108, 110 potentially resulting in an accidental needle stick injury. Needle retraction subassembly 100 is retained in the detachable activation mechanism 1202 housing and disposed of after insertion.

Infusion system 1200 also includes detachable activation mechanism 1202 that is configured to insert indwelling infusion catheter 104 and CGM sensor 106, by way of introducer/insertion needles 110, 108, respectively, into the subcutaneous tissue of a user's skin two insertion sites normal to the user's skin. Specifically, detachable activation mechanism 1202 is configured to drive cartridge assembly 102 from a telescoping position on the top surface of device 600, wherein infusion catheter 104 and CGM sensor 106 are in a retracted position to (2) an advanced position within device 600, wherein infusion catheter 104 and CGM sensor 106 are in a deployed position embedded into the subcutaneous layer of the user's tissue (after insertion). Detachable activation mechanism 1202 is further configured to insert infusion catheter 104 and CGM sensor 106. This is described in more detail below. The infusion system 1200 is described in detail in the exploded view in FIG. 20.

Device 600 is a wearable apparatus, system or pod for diabetes management in which continuous glucose monitoring (CGM), insulin delivery and control functionality are provided to ensure insulin is delivered at very precise rates and has the capability of detecting occlusions in real time. In operation, device 600 is applied by opening a sterile packaging, filling the reservoir with insulin, priming the fluid path, removing the adhesive backing, sticking infusion system 1200 to the desired body location, pushing a button (also called button assembly), removing and disposing the detachable activation mechanism 1202. Removing detachable activation mechanism 1202 components that are necessary for device 600 activation and needle insertion, but not required for infusion or sensing offers the benefit of a smaller, lower profile, more comfortable and discrete wearable device 600 for the user. This is described in more detail below.

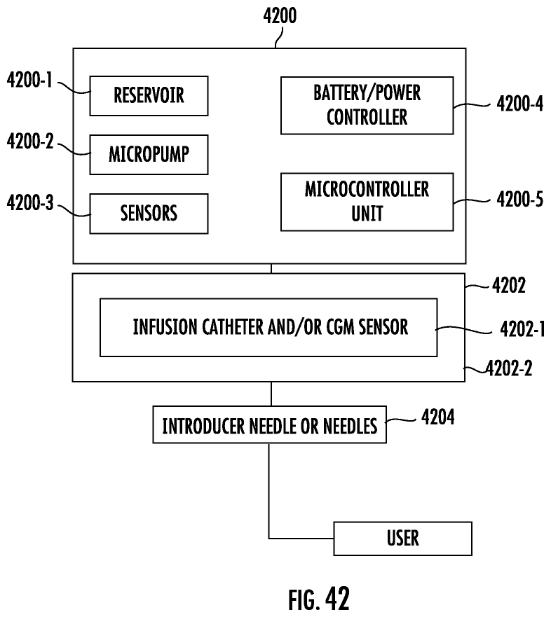

As described in detail below, cartridge assembly 102 includes infusion catheter 104 and CGM sensor 106 as shown. Needle retraction assembly 100 comprises several components including introducer needles 108, 110. The embodiment described herein utilizes two separate introducer needles for introducing both an infusion catheter and CGM sensor, but the same needle retraction assembly can be used for a single site infusion catheter or CGM sensor architecture which uses one introducer needle for an insertion mechanism that inserts an infusion catheter or a CGM sensor alone (either). An example retraction assembly with a single introducer needle for introducing an infusion catheter is shown in FIG. 41.

Introducer needles 108, 110 are preferably constructed of steel but it may be any rigid material known to those skilled in the art. CGM sensor 106 is constructed of many materials as known to those skilled in the art. Infusion catheter 104 for this mechanism can be the untipped (dull) steel indwelling needle however the preferred embodiment is a flexible polymeric catheter. This automatic needle retraction assembly is an alternative to a manual needle removal design. The advantage of the automatic retraction mechanism are convenience from fewer steps for the user and automatic needle tip shielding to prevent accidental needle stick injury during removal and disposal of the used insertion mechanism.

Device 600 incorporates, among other elements (as described below), a micropump as known to those skilled in the art that can be used for pumping fluid, valves used for regulating flow, actuators used for moving or controlling the micropump and valves and/or sensors used for sensing pressure and/or flow. The micropump may be used to infuse the insulin or other fluidic medication (also known as medicament) to the user. Medication may include small molecule pharmaceutical solutions, large molecule or protein drug solutions, saline solutions, blood or other fluids known to those skilled in the art. Insulin is an example of fluid that is described in this application. However, micropump may be used in other environments known to those skilled in the art.

Device 600 also includes a housing that houses a reservoir, a microcontroller unit (MCU) (not shown) and a battery and power controller (not shown). Device 600 also includes cartridge assembly 102. The reservoir is configured to receive and store insulin for its delivery over a course of about three days, or as needed. However, reservoir size may be configured for storing any quantity of fluid as required. The micropump fluidly communicates with reservoir to enable infusion as needed. CGM, as known to those skilled in the art, tracks patient glucose levels and permits those levels to be used in algorithms that control flow rate. The micropump may be a MEMS device or other electro-mechanical device known to those skilled in the art. MCU controls the operation of the micropump to deliver insulin through an insulin catheter from the reservoir at specific doses, i.e., flow rates over specified time intervals, based on CGM data converted to desired flow rate via control algorithms. The battery and power controller controls the power to the MCU and the micropump to enable those components to function properly as known to those skilled in the art. The CGM is powered by battery and the power controller through the MCU.

Cartridge assembly 102 is configured to fit within a cartridge (insertion) compartment opening in the top housing and a channel extending through top housing of device 600. The cartridge (insertion) compartment opening in the top of the housing enables access for the cartridge assembly 102 so it can drive infusion catheter 104 and CGM sensor 106 into the subcutaneous tissue of a user.

Upon needle insertion, the opening is closed off, so the housing provides some sealing properties and creates a continuous surface. Detachable activation mechanism 1202 compartment is outside of the hermetically sealed compartment that contains the pump and electronics of device 600 for delivering insulin so water ingress can occur without affecting device 600 performance. The amount of water ingress is limited by minimizing the volume of empty space in order to reduce the wetting nuisance that can occur after a user's swim or shower.

In brief, as indicated above, cartridge assembly 102 is configured to move from (1) a telescoping position above the top surface of device 600, wherein integrated infusion needle and CGM sensor subassembly 102 is in a retracted position to (2) an advanced position within device 600, wherein infusion catheter 104 and CGM sensor 106 of cartridge assembly 102 are in a deployed position embedded into the subcutaneous layer of the user's tissue (after insertion). This is described in more detail below. Cartridge assembly 102 is an example of a carrier or carrier assembly for carrying an infusion catheter and/or a CGM sensor for example (or other analyte sensor), Cartridge assembly 102 may incorporate a custom designed CGM sensor subassembly or an off the shelf CGM sensor subassembly (commercially available) which includes the sensor chassis, introducer needle, sensor and electrical components or slightly modified versions of these subassemblies.

As indicated above, FIGS. 1-3 depict needle retraction subassembly 100 assembled onto cartridge subassembly 102 before use, after introducer needles 108, 110 insertion, but before the needle retraction is triggered (cross-section) and after needle retraction (cross-section) respectively. The other device components are hidden for clarity.

FIG. 4 depicts an exploded view of needle retraction assembly 100.

Assembly 100 includes release lever 112, release housing 114, lever hinge dowl 116, retraction housing 118, needle hubs 120, introducer needles 108,110, needle retraction springs 122,124 release housing cover 126, and housing screw 128.

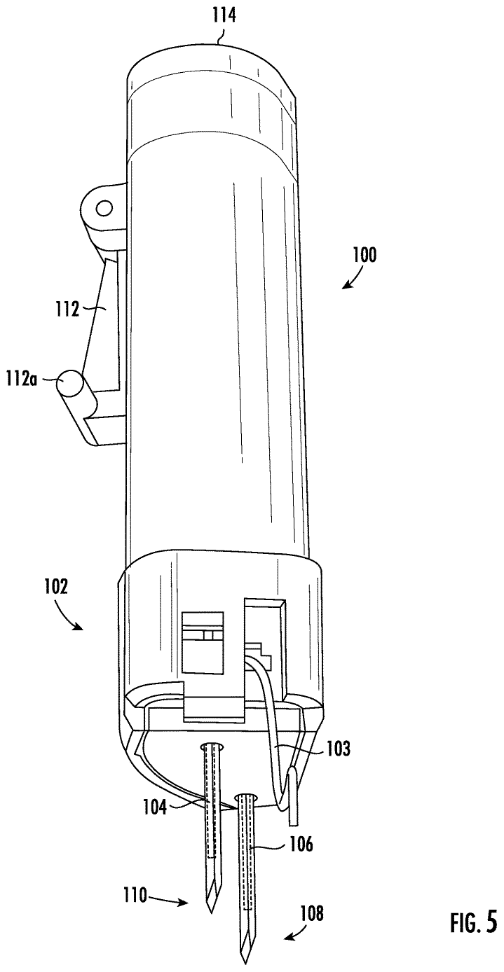

FIG. 5 depicts cartridge assembly 102 and needle retraction assembly 100 with infusion catheter 104 made of PEEK and CGM sensor 106. Cartridge assembly 102 is connected to the insulin (or other medication) fluid path and CGM electronics and assembled in the pod before being assembled into the detachable activation mechanism 1202. These are the components driven downward by detachable activation mechanism 1202 to insert CGM sensor 108 and catheter 104 into the tissue.

FIG. 6 depicts catheter and sensor cartridge 102 fluidically and electrically connected to the device 600. This is the configuration of the device 600 before it is assembled into detachable activation mechanism 1202. In this configuration, introducer needles 108, 110 are coupled with catheter 104 and sensor cartridge 106 in the pre-activation configuration.

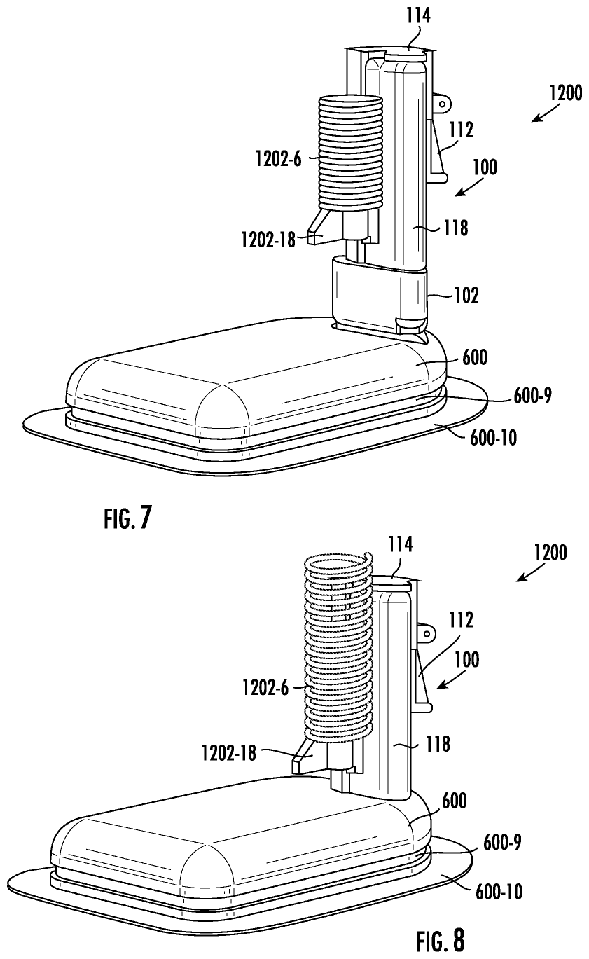

In FIGS. 7 and 8, when the detachable activation mechanism 1202 is activated, insertion spring 1202-6 (and shown in FIG. 20) is released forcing insertion rod 1202-18 downwards. Insertion rod 1202-18 (and shown in FIG. 20) forces needle retraction assembly 100 and cartridge assembly 102 downwards thereby inserting sensor 106 and infusion catheter 104 into the tissue. Some components are not shown for clarity.

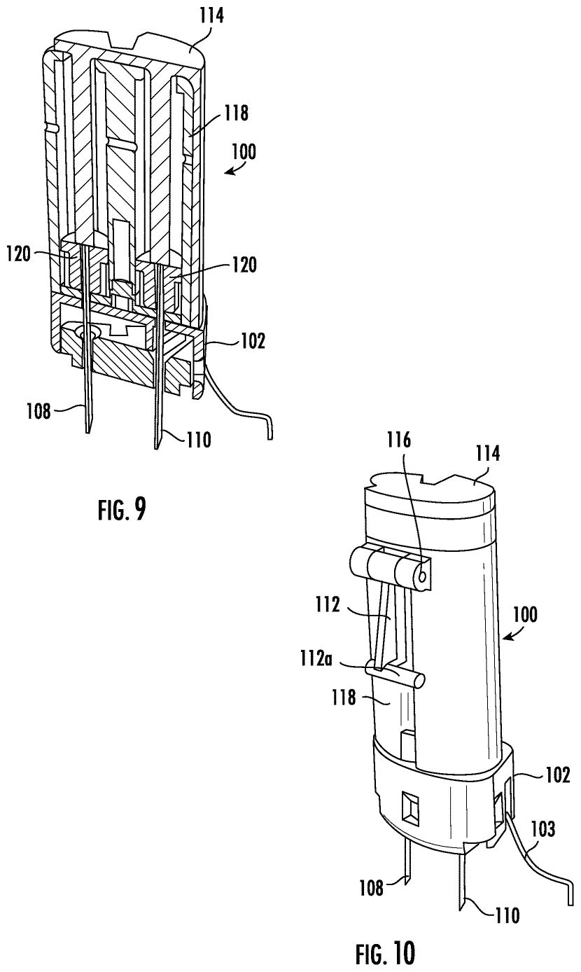

FIGS. 9-11 depict various views of retraction mechanism assembly 100 mounted on cartridge assembly 102. In the pre-activation configuration, release housing 114 forces introducer needles 108,110 in a downward position and compresses retraction springs 122,124. Release lever 112 is attached with a hinge to release housing 114 and configured to detachably couple the release housing 114 from retraction housing 118. When the housings are coupled, introducer needles 108,110 are exposed for insertion, retraction springs 122,124 are loaded, and the assembly 100 is ready for insertion.

FIGS. 12 and 13 depict cross sectional views, in part, of infusion system 1200 with device 600 in a pre-activation state (wherein introducer needles 108,110 are within device 600) and an intermediary state during insertion, respectively. Note that release lever 112 rotational angle is maintained during insertion in order to couple the release and retraction housings 114,118, respectively together in order to hold introducer needles 108, 110 outwards for insertion.

Detachable activation mechanism housing 1202-2 includes release lever rib 1202-2a that pushes inward on release lever 112 to keep the release catch 112b locked. Release lever rib 1202-2a changes into a ramp 1202-2a1 (surface).

Housing 1202-2 also includes projection 1202-2b extending outwardly along the internal surface. Projection 1202-2b is angled to form cam surface 1202-2b1 to engage boss 112a of release 112 as described in more detail below. Cam surface 1202-2b1 and ramp 1202-2a1 together define a slot 1203 to receive boss 112a of release lever 112 as it slides into slot 1203. As stated earlier, FIG. 12 depicts device 600 in the pre-activation configuration (i.e., introducer needles 108, 110 within device 600).

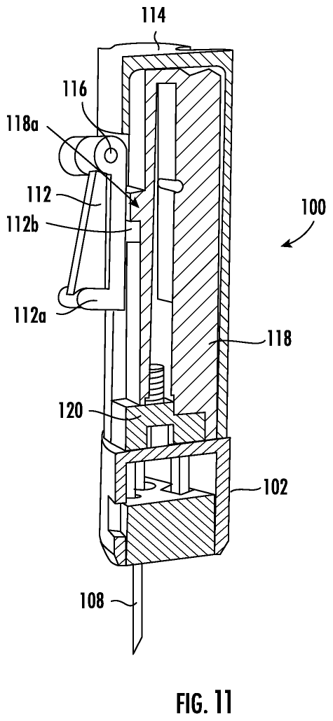

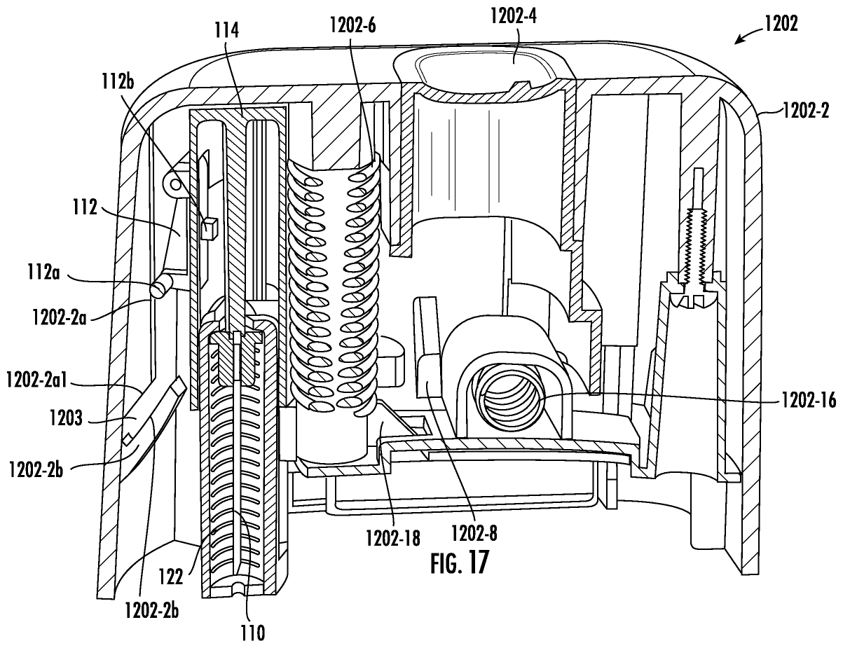

FIG. 13 depicts introducer needles 108,110 half way through insertion with extended boss 112a of release lever 112 that slides on release lever rib 1202-2a on the inside of detachable activation mechanism housing 1202-2 of detachable activation mechanism 1202. In both positions, rib 1202-2a holds release lever 112 inward such that release lever catch 112b (FIG. 11) is engaged with snap 118a (FIGS. 11, 16) on the retraction housing 118.

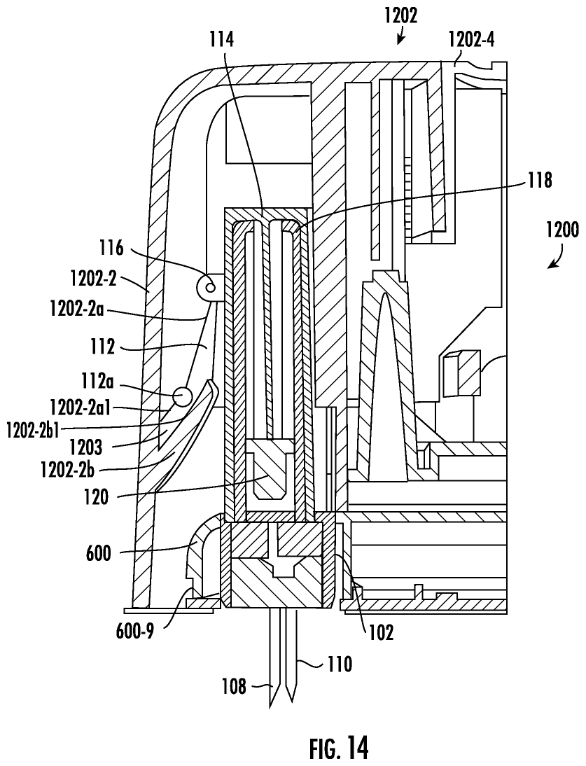

FIG. 14 depicts a cross sectional view, in part, of infusion system 1200 wherein introducer needles 108,110 are in a deployed configuration (but needles 108,110 have not been retracted). Detachable activation mechanism housing 1202-2 release lever rib 1202-2a leads to ramp 1202-2a1. When detachable activation mechanism 1202 housing is removed after activation, release lever 112 slides down rib 1202-2a into slot 1203 against cam surface 1202-2b1 of projection 1202-2b, thereby rotating to a position which decouples release lever catch 112b from snap 118a on the retraction housing 118 which decouples release and retraction housings 114,118 respectively.

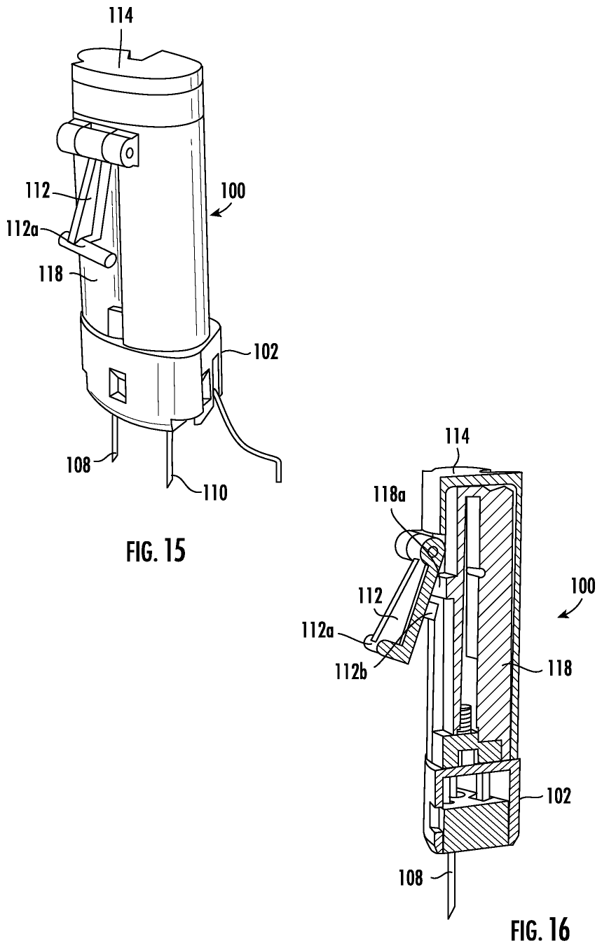

FIGS. 15 and 16 depicts views of needle retraction assembly 100. In particular, FIGS. 15 and 16 depict needle retraction assembly 100 with lever catch 112b disengaged just before springs 122,124 will retract introducer needles 108,110.

FIG. 17 depicts a cross-sectional view of detachable activation mechanism 1202 portion of infusion system 1200 in the post activation configuration with needle retraction springs 122,124 expanded, introducer needles 108,110 retracted and device 600 removed. In this configuration the needle tips are shielded to prevent accidental needle stick injury. The tips are misaligned with the exit holes in the mechanism 1202 housing so if a force is applied to the housing the needles are not exposed. Example components of infusion system 1200 in exploded configuration are shown in FIG. 20 and described below.

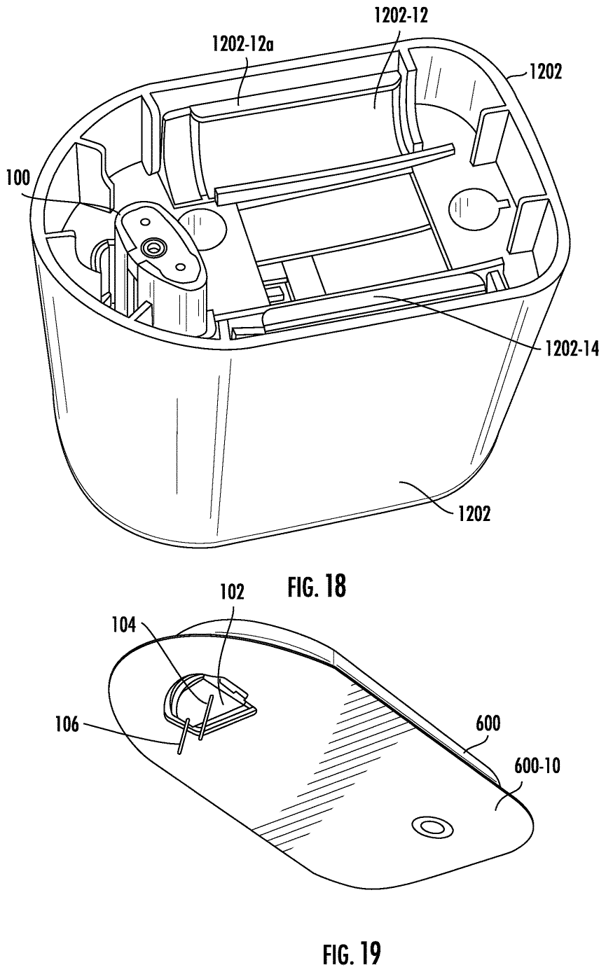

FIG. 18 depicts a bottom view of the post-activation detachable activation mechanism 1202 showing among other components, the left and right release arms 1202-12, 1202-14 with flanges 1202-12a, 1202-14a, respectively.

FIG. 19 depicts device 600 for delivering insulin or other medication in the wear configuration after sensor 106 and catheter 104 have been inserted, introducer needles 108,110 have been retracted and the detachable activation mechanism 1202 removed.

FIG. 20 depicts a view of example components of the infusion system 1200 in an exploded configuration. Device 600 for delivering insulin includes top housing 600-2, baseplate 600-4, integrated infusion needle and CGM sensor 600-6, insertion mechanism tubing 104-8 and adhesive patch 600-10. Device 600 as shown and described herein is configured to be mounted to the user (and remain on the user by way of adhesive patch 600-10). Device 600 includes groove 600-9 (FIGS. 6-8) along the lower periphery thereof that is defined by housing 600-2 and baseplate 600-4 in an assembled configuration. Groove 600-9 is used to receive flanges 1202-12a and 1202-14a of left release arm 1202-12 and right release arm 1202-14 of detachable activation mechanism 1202 as described in more detail below.

Detachable activation mechanism 1202 includes detachable mechanism housing 1202-2, activation button (also referred to as activation button assembly) 1202-4, insertion spring 1202-6, activation beam 1202-8, insertion mechanism inner housing 1202-10, left release arm 1202-12, right release arm 1202-14, release spring 1202-16, insertion rod 1202-18 and a number of screws for assembling these components (shown but not numbered). Button 1202-4 is a trigger or trigger mechanism that functions as a trigger to activate detachable activation mechanism 1202 as described in detail below. However, elements or components other than a button may be used as a trigger as known to those skilled in the art.

Cartridge assembly 102 includes needle cartridge 102-2, needle cartridge or needle cartridge cover 102-4 and cartridge lock snap 102-7. Cartridge cover 102-4 slides within cartridge 102-2 and is fully integrated together. Introducer needles 106, 108 are press fit or glued through an opening in needle cartridge cover 102-4 to extend beyond the bottom surface thereof. Tubing 600-8 is fluidly connected to infusion catheter 104 as known to those skilled in the art and tubing 600-8 extends through side rear wall of cartridge cover 102-4 for fluid connection to a filled reservoir via a micropump. A lock snap or similar component as known to those skilled in the art is configured to act as a locking mechanism to lock the cartridge assembly 102 down and force introducer needles 108, 110 in a user's skin. (The needle cartridge lock snap engages with a coupling ledge as part of top housing 600-2).

As indicated above, needle cartridge assembly 102 is configured to slide or move through channel 600-3 within device 600 from a telescoping position to a position fully integrated within device 600 whereby introducer needles 108, 110 are inserted into the subcutaneous tissue of the user. It is the construction of slidable needle cartridge assembly 102 and its operation with respect to device 600 offers the benefit of or facilitates a low profile configuration of device 600 while creating a hermetic seal to prevent leaks around the channel.

To this end, needle cartridge assembly 102 is configured to slide through an opening leading to channel 600-3. An example of a cartridge assembly is disclosed in published application PCT/US23/11318 (WO2023/141316), filed Jan. 23, 2023, entitled “Detachable Activation Mechanism and Needle Cartridge Assembly For Inserting Infusion Needle and CGM Sensor” and published application PCT/US23/11320(W02023/141318 ), filed Jan. 23, 2023, entitled “Detachable Activation Mechanism and Cartridge Assembly For Inserting Dual Introducer Needles” which are both incorporated by reference herein. Cartridge assembly 102 is an example of a carrier or carrier assembly for carrying an infusion catheter and/or analyte sensor.

In practice, device 600 adhesive backing is removed from device 500 for delivering insulin and detachable activation mechanism 1202 and applied to a user's body. After application, infusion system 1200 is in a pre-activation configuration, device 600 for delivering insulin is securely attached to and within the detachable activation mechanism 1202 by way of retention flanges 1202-12a, 1202-14a on right and left release arms 1202-12, 1202-14, respectfully. These flanges are biased outwardly by release spring 1202-16. In the locked position, flanges 1202-12a, 1202-14a engage groove 600-9 of device 600 to secure device 600 within detachable activation mechanism 1202. Activation beam 1202-8 holds insertion rod 1202-18 in an upward position and spring 1202-6 loaded. Button ledges 1202-4a FIG. 20) on button 1202-4 lock the release arms 102-12,102-14 in the closed position so that flanges 102-12a, 102-14a within groove 600-9 lock the device 600 in detachable activation mechanism 1202. (The opposing side of button 1202-4 also has a ledge, but it is now shown in FIG. 20.) The button ledges interfere with release arms 102-12, 102-14 before activation.

When activation button 1202-4 is pressed, a ramped surface on button 1202-4 translates downward, causing the activation beam 1202-8 to rotate, freeing release of insertion rod 1202-18. Under load from spring 1202-6, insertion rod 1202-18 moves downward, thereby forcing cartridge assembly 102 downward as well into channel 600-3 in device 600. Insertion rod 102-18 pushes needles 108,110 into an inserted position within the subcutaneous tissue of the user. Simultaneously, when button 1202-4 translates downward by force from the user, button ledges 1202-4a of button 1202-4 (of detachable activation mechanism 1202) move into a position without interference from release arms 1202-12, 1202-14 and spring 1202-16 causes flanges 1202-12a, 1204-14a of release arms 102-12,102-14 to move outwardly, thereby moving flanges 1202-12a, 1204-14a out of a groove 600-9 (also called indentation), releasing device 600. In a post, a post activation configuration, but before detachable actuation mechanism 1202 has been removed, retention flanges 1202-12a, 102-14a have moved outwardly, allowing device 600 to detach from detachable activation mechanism 1202. In short, downward button translation also triggers the release of detachable activation mechanism 1202 from device 600 and retraction mechanism 100 is triggered to automatically retract and retain the introducer needles inside the needle retraction assembly 2100 (part of detachable activation mechanism 1202) and shield the introducer needles described herein from the user.

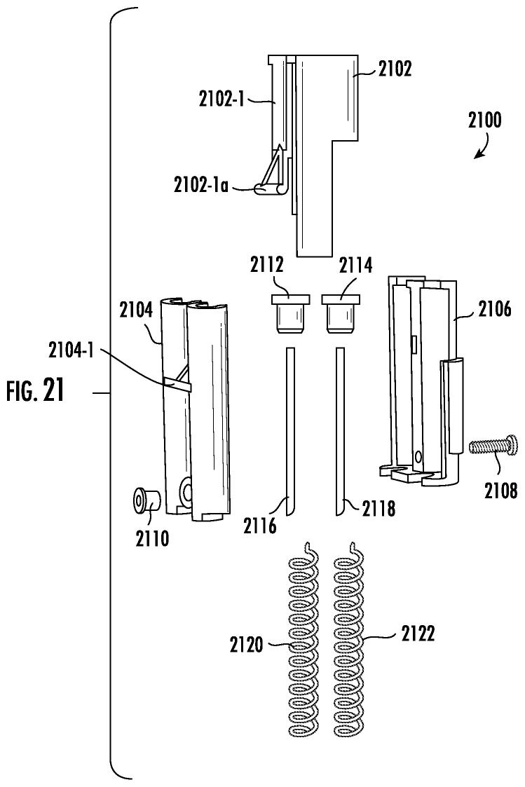

FIG. 21 depicts an exploded view of the components of another example needle retraction assembly 2100 in FIG. 21. Similar to needle retraction assembly 100, needle retraction assembly 2100 is part of infusion system 1200. Infusion system 1200 also includes the same components such as device 600 for delivering insulin and detachable mechanism 1202. Therefore, reference to such components are the same.

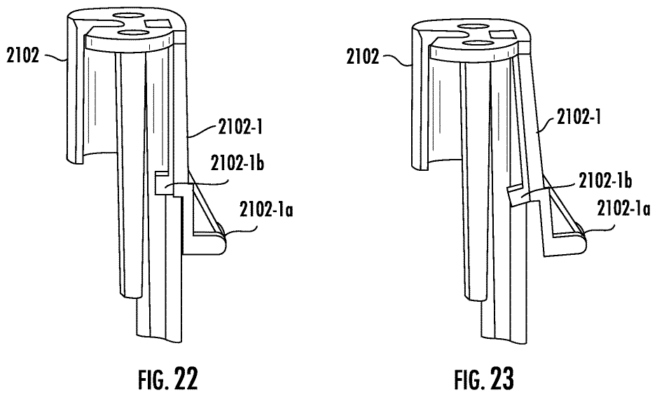

Needle retraction assembly 2100 includes release housing 2102 with a release lever 2102-1, retraction housing lever side 2104, retraction housing screw side 2106, retraction housing screw 2108, threaded insert 2110, introducer needle hubs 2112, 2114, introducer needles 2116, 2118 and needle retraction springs 2120, 2122. Retraction housing screw 2108 is configured to enter a bore in lower portion of retraction housing screw side 2012 and mate and thread with threaded insert 2110 and needle retraction springs 2120, 2122. In this example retraction assembly 2100 (compared to the example above), release lever 2102-1 now integrates a lever hinge dowl as described ending in boss 2102-1a (similar to boss described above). That is, release lever 1202-1 is part of release housing 2102 as a single injection molded part. Instead of a hinge rotating to trigger needle retraction as described above, release lever 1202-1 actually flexes. This is best depicted in FIGS. 22 and 23 wherein release housing 2102 is show in non-flexed and flexed configurations, respectively, In the flexed configuration or state, needle retraction is triggered.

In this example retraction assembly 2100, retraction housing lever side 2104 and retraction housing screw side 2106 replace the retraction housing 118 in the other embodiment retraction assembly 100 above. This is described in more detail below.

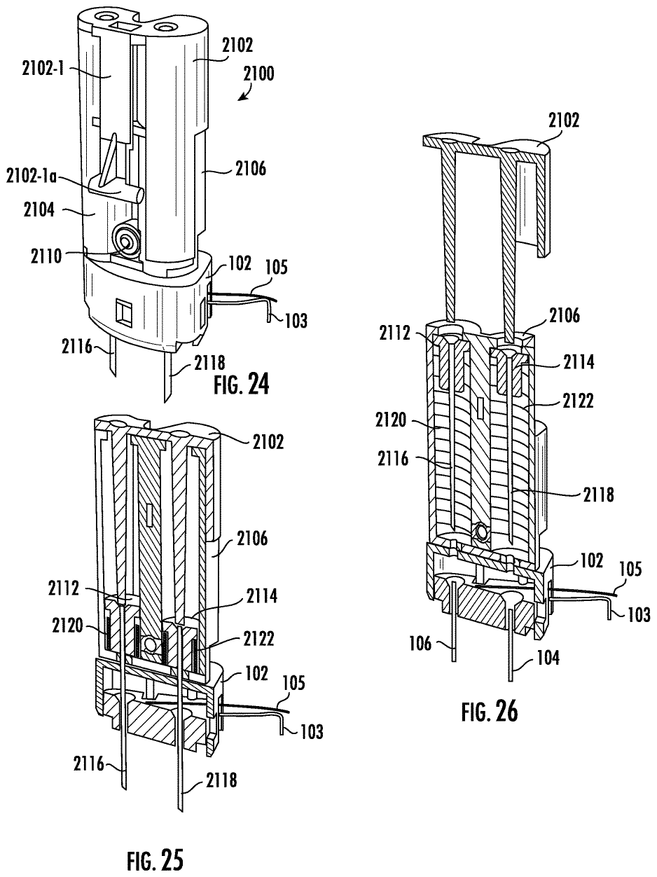

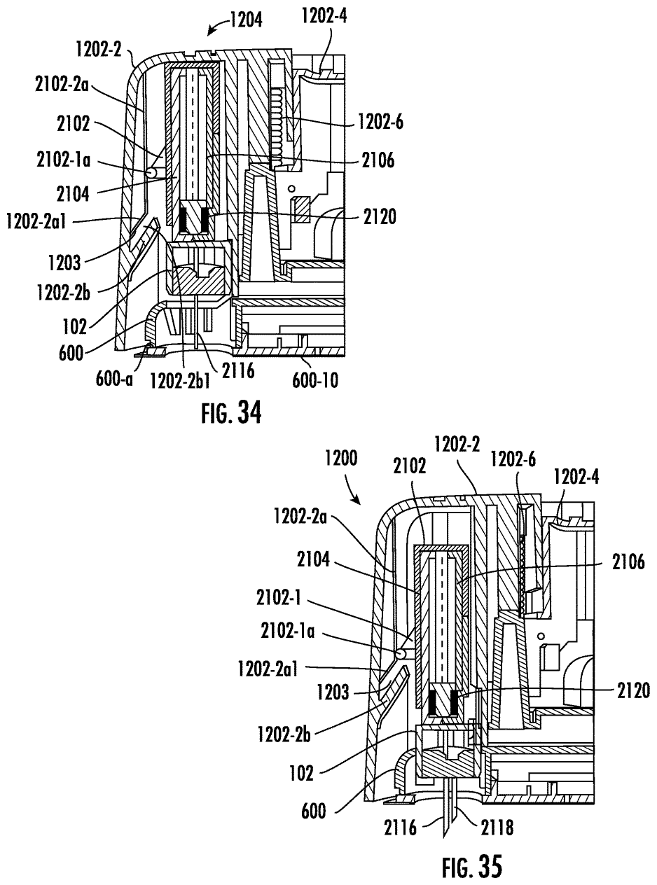

FIGS. 24-25 depict various views of another example needle retraction assembly 2100 for device 600 for delivering insulin or other medication (of an infusion system) in a pre-activation configuration and after needle insertion, but before needle retraction is triggered. As described above, needle retraction assembly 2100 is part of detachable activation mechanism 1200 shown in FIGS. 34-36 and 39 discussed below). Needle retraction assembly 2100 is mounted on cartridge assembly 102 as described above and it incorporates an infusion catheter and continuous glucose monitoring (CGM) sensor to be advanced into the subcutaneous layer of the tissue as described in more detail below. In this configuration, release housing 2102 is advanced downwardly against introducer needle hubs 2112, 2114 to align with attached or embedded needles 2116, 2118 extending from cartridge assembly 102.

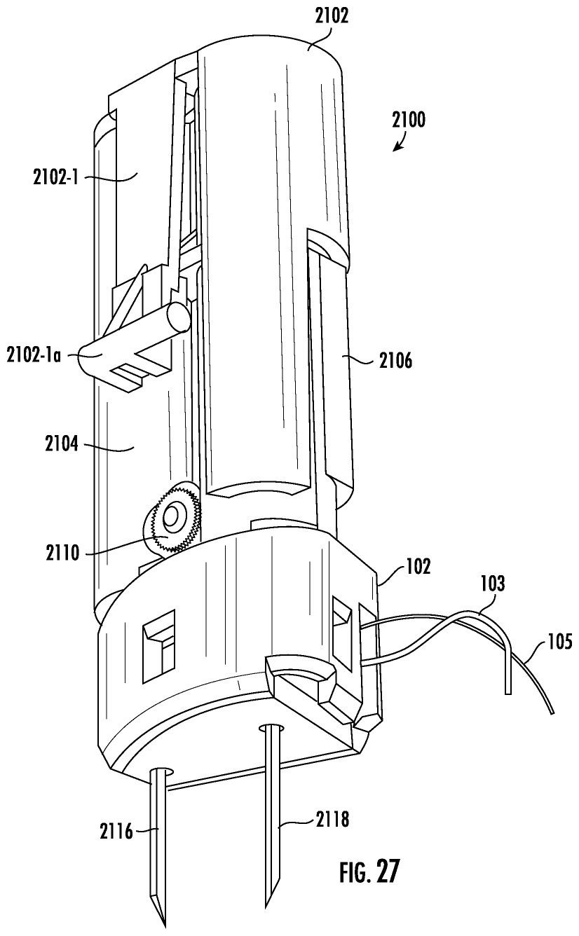

FIG. 26 depicts a cross-sectional view of the example needle retraction assembly 2100 in FIG. 21 after needle retraction. In this configuration, release lever 2102-1 has flexed outwardly as boss 2102-1a slides down a ramp 1202-2a1 (described below) into slot 1203, decoupling the release lever catch 2102-1b from the snap 2104-1 on retraction housing side 2104. Thus, springs 2120, 2122 are decompressed with introducer needle hubs 2112, 2114 in a fully retracted position inside retraction housing sides 2104, 2106 along with introducer needles 2120, 2122. Importantly, needles are configured as U-shaped (open channels) to avoid interfering with the wiring 2124 for the CGM and tubing 2126 as needles 2120, 2122 are withdrawn.

Cartridge assembly 102 and its mounting and functions are the same as described above with respect to cartridge assembly 102. Therefore, cartridge assembly 102 will not be described here. In addition, device 600 (also referred to as a delivery device 600 or pod) is a component of infusion system 1200 in FIG. 34.

FIG. 27 depicts an enlarged perspective view of the needle retraction assembly 2100 in FIG. 21, in assembled form mounted on cartridge assembly 102.

The infusion catheter may be PEEK or other material known to those skilled in the art. Cartridge assembly 102 is connected to the insulin (or other medication) path and the CGM electronics and assembly in device 600 before assembling into detachable activation mechanism 1202.

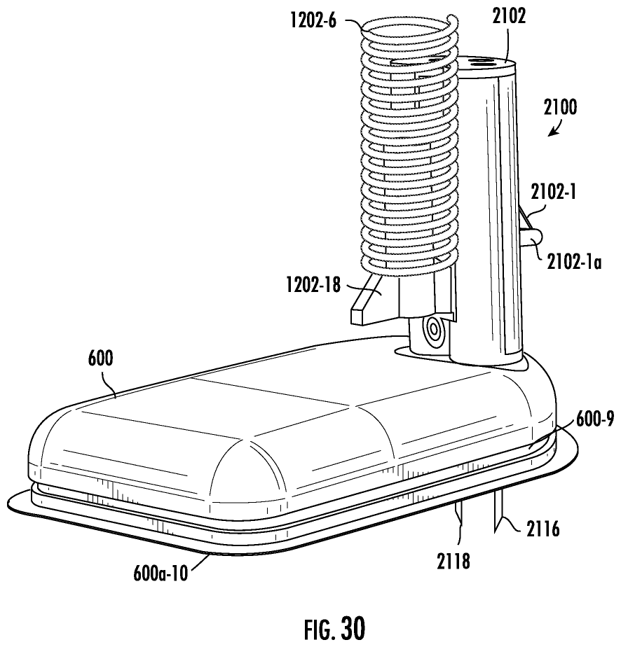

FIG. 28 depicts a perspective view of the needle retraction assembly in FIG. 21 and cartridge assembly 102 fluidically and electrically connected to the device 600 for delivering insulin (or other medication) of infusion system 1200 (before device for delivering insulin is assembled into a detachable activation mechanism 1202). In this configuration, introducer needles are coupled with the cartridge assembly in pre-activation configuration. FIG. 29 depicts a rear view of the needle retraction assembly and cartridge assembly in FIG. 28.

FIG. 30 depicts a rear perspective view of the needle retraction assembly in FIG. 21 in assembled form, and cartridge assembly after introducer needle insertion. When detachable activation mechanism 1202 is activated, insertion spring 1202-6 is released forcing insertion rod 1202-18 downwards. Insertion rod 1202-18 forces needle retraction assembly 2100 and cartridge assembly 102 downwards thereby inserting the sensor and catheter into a user's tissue. Certain components are not shown in FIG. 30 for clarity.

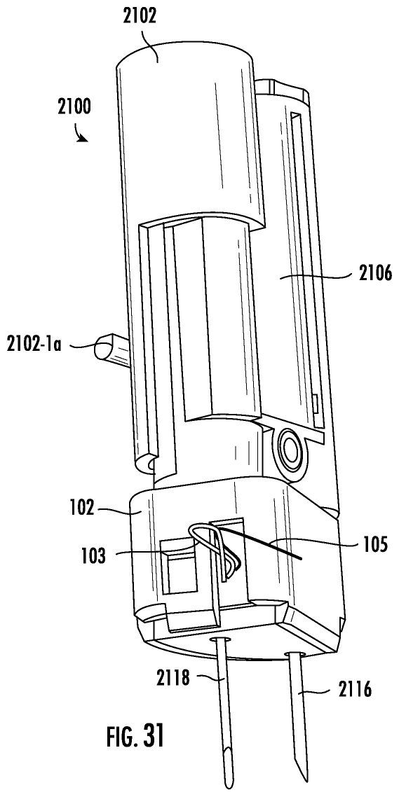

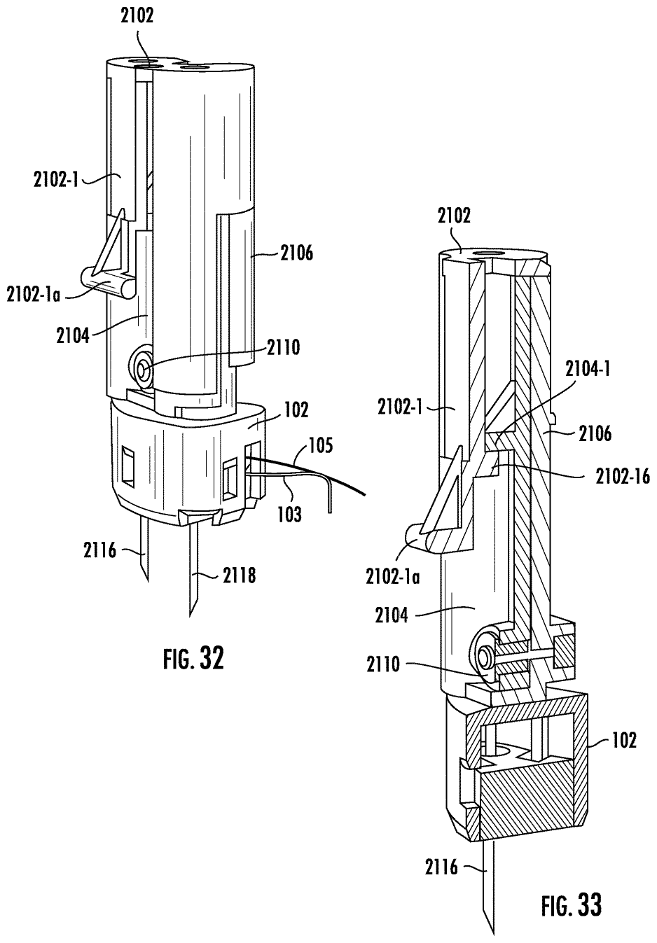

FIGS. 31 and 32 depict a rear perspective view and a front perspective view of the needle retraction assembly mounted on the cartridge assembly in FIG. 29 in which the introducer needles are exposed for insertion. FIG. 33 depicts a cross sectional view of the needle retraction in FIG. 32. In pre-activation configuration, release housing 2102 maintains introducer needles 2116, 2118 in a downward position and compresses retraction springs 2120, 2122.

Release lever catch 2102-1b is attached to a flexible beam of release housing 2102 and detachably couples release housing 2102 to retraction housing sides 2104, 2106. When housings 2102 and 2104, 2106 are coupled, introducer needles 2116, 2118 are exposed for insertion, retraction springs 2120, 2122 are loaded and cartridge assembly 102 is ready for insertion (into device 600). Release lever rib 1202-1 ends with ramp 1202-1a to receive boss 2102-1a of release lever 2102-1. Release lever 2102-1 rotational angle is maintained during insertion in order to couple the release and retraction housings 2102 and 2104, 2106 together to hold introducer needles 2116, 2118 outwards for insertion. Release lever rib 1201-1 in detachable activation mechanism housing 1202-2 pushes inward on release lever 2102-1 to keep release catch 2102-1b locked.

FIG. 34 depicts a cross sectional view of the infusion system 1200 in which device 600 is in a pre-activation configuration. FIG. 35 depicts infusion system 1200 in which introducer needles 2116, 2118 are in a halfway insertion configuration with release lever 2102-1 that slides on release lever rib 1201-1 of detachable activation mechanism 1202. Release lever rib 1202-1 is configured to push inward on release lever 2102-1 of needle retraction assembly 2100 to keep the release catch 2102-1b locked. In both positions, rib 1202-1 hold release lever 2102-1 inward such that release lever catch 21-2-1b is engaged with snap 2104-1 (FIG. 33) on retraction lever housing side 2104.

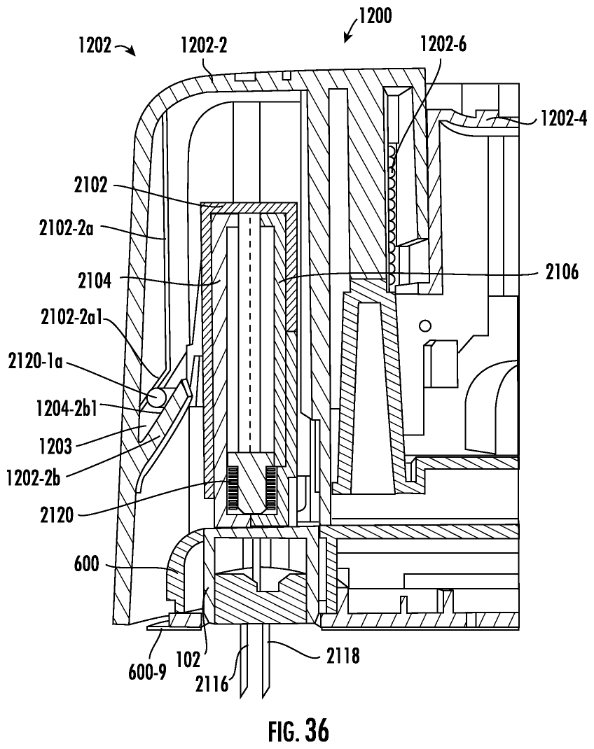

FIG. 36 depicts an enlarged cross sectional view the infusion system in FIG. 35. As indicated above, release lever rib 1202-1 ends with ramp 1202-1a to receive boss 2102-1a of release lever 2102-1. When detachable activation mechanism housing 1202-2 is removed after activation, boss 2102-1a of release lever 2102-1 slides down rib 1202-2a and then along ramp 1202-2a1 into slot 1203, against cam surface 1202-2b. Thus, lever 2102-1 flexes to a position which decouples release lever catch 2102-1b from snap 2104-1 on retraction lever housing side 2104 thereby decoupling release and retraction housings 2102 and 2104, 2106.

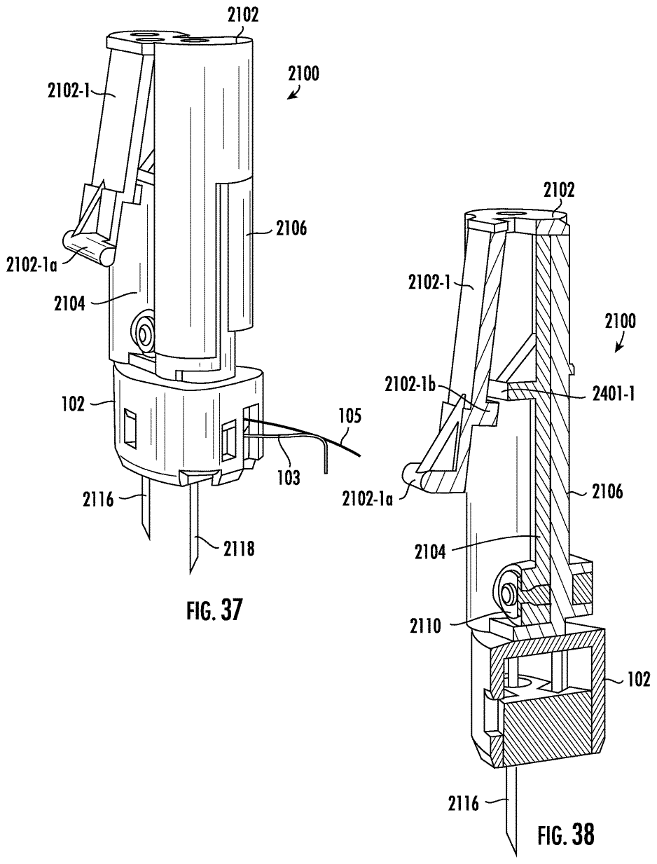

FIG. 37 depicts a perspective view of the needle retraction assembly of FIG. 21 with a lever catch disengaged. FIG. 38 depicts a cross sectional view of the needle retraction assembly in FIG. 37. In particular, needle retraction assembly 2100 is shown with lever catch 2102-1b disengaged just before springs 2120, 2122 would retract introducer needles 2116, 2118.

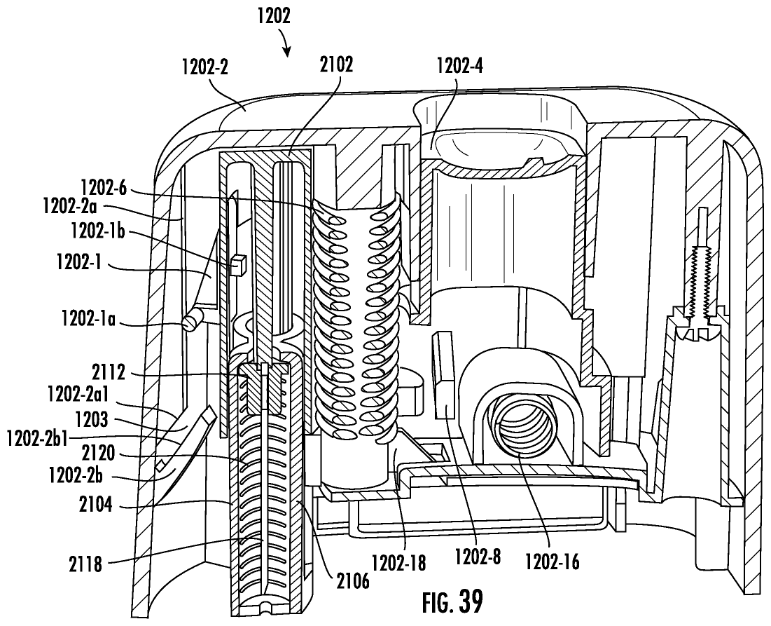

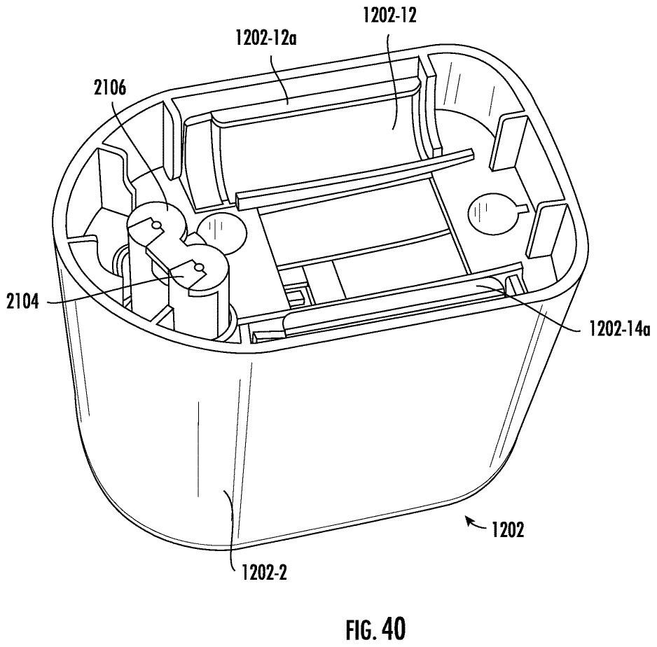

FIG. 39 depicts a cross-sectional view of the activation mechanism portion of the infusion system shown in FIG. 34 in which the detachable activation mechanism is in a post activation configuration with needle retraction springs 2120, 2122 expanded, needles 2116, 2118 retracted and device 600 removed. In this configuration, needle tips are shielded to prevent accidental needle stick injury. The tips are misaligned with the exit holes in the housing so if a force is applied to the retraction housing, needle tips are not exposed and cannot harm the user.

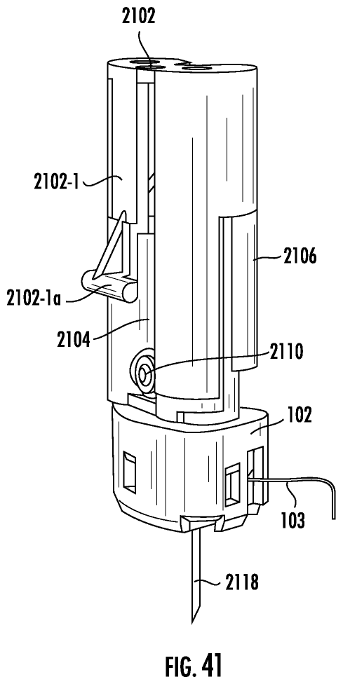

FIG. 40 depicts a bottom view of the detachable activation mechanism in FIG. 39 in post activation configuration. In particular, needle retraction assembly contains or houses the used introducer needles 2116, 2118 and is retained as part of detachable activation mechanism 1202 and disposed of after use.

FIG. 41 depicts a perspective view of another example needle retraction assembly. That is, this is an example retraction assembly with a single introducer needle for introducing an infusion catheter (only). The exact same components are employed within this retraction assembly with the same functionality. Therefore, the same reference numeral have been used. The only different is that there is only one introducer needle 2118 shown for introducing an infusion catheter 104. However, this introducer needle 2118 can alternatively be used for introducing a CGM sensor.

FIG. 42 depicts a block diagram of selective example components of the infusion system 1200 above including components of device 600 for delivering insulin or other medication, needle retraction assembly 100 or 2100 and cartridge assembly 102 described above. In this FIG. 42, device 600 has been renumbered as device 4200, needle retraction assembly 100 or 2100 is renumbered as assembly 4204 and cartridge assembly 102 renumbered as cartridge assembly 4202.

Device 4200 incorporates several components or modules (not shown) within a housing in the fluidic pathway including reservoir 4200-1 for storing the insulin, micropump 4200-2 for pumping the insulin, sensors 4200-3 (e.g., pressure) for sensing various parameters in the system and user and tubing connecting infusion needle 1802-1 to reservoir 4200-1 within cartridge assembly 4202. Device 4200 also includes microcontroller unit (MCU) 4200-4 and battery and power controller 4200-5. Device 4200 further incorporates insertion needle 4200-6 (described herein) along with infusion catheter 4200-7 and CGM sensor 4200-8.

CGM, as known to those skilled in the art, tracks user glucose levels and permits those levels to be used in algorithms that control flow rate. MCU 4200-4 controls the operation of micropump 4200-2. Cartridge assembly 4202 includes infusion catheter and/or and CGM sensor 4202-2 subassembly are shown. That is, one or both of these (catheter and sensor) may be used with one or two introducer needles as described herein.

Reservoir 4200-1 is configured to receive and store insulin for its delivery over a course of about three days, or as needed. However, reservoir size may be configured for storing any quantity of fluid as required.

MCU 4200-5 electronically communicates with sensors 4200-3 and micropump 4200-2 as well as the CGM sensor 4204-1, as the monitoring components. Among several functions, MCU 4200-5 operates to control the operation of micropump 4200-2 to deliver insulin through insulin catheter 4204-1 from reservoir 4200-1 at specific doses, i.e., flow rates over specified time intervals, based on CGM data converted to desired flow rate via control algorithms.

Battery and power controller 4200-4 controls the power to MCU 4200-5 and micropump 4200-2 to enable those components to function properly as known to those skilled in the art. CGM sensor 4200-8 is powered by battery and power controller 4200-4 through MCU 4200-5.

Retraction assembly 4204 includes, among other components, one or two introducer needles for introducing an infusion catheter and/or a CGM sensor as shown.

The components of device 4200, cartridge assembly 4202 and needle retraction assembly 4204 shown in FIG. 42 are only a few components. Those skilled in the art know that device 4200, cartridge assembly 4204 and retraction assembly 4202 may include additional components or less components.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. An infusion system comprising:
a device configured to deliver medication to a user through an infusion catheter, wherein the device includes a housing configured to receive the infusion catheter and to maintain the infusion catheter in the user after insertion; and
a detachable activation mechanism for deploying the device for delivering medication to the user, wherein the detachable activation mechanism comprises a trigger and a retraction mechanism that includes an introducer needle for introducing the infusion catheter into the user,
wherein the detachable activation mechanism is configured to releasably connect to the housing,
wherein the detachable activation mechanism is configured to insert the infusion catheter into the user, automatically release the detachable activation mechanism from the housing responsive to activation of the trigger and automatically retract the introducer needle into the needle retraction assembly to shield the needle upon the automatic release of the detachable activation mechanism.

2. The infusion system of claim 1 further comprising a cartridge configured to move with respect to the housing in response to movement of the needle retraction assembly and insert the infusion catheter into the user.

3. The infusion system of claim 1 wherein the medication is insulin.

4. An infusion system comprising:
a device configured to deliver medication to a user through an infusion catheter, wherein the device includes a housing configured to receive the infusion catheter and to maintain the infusion catheter in the user after insertion;
the detachable activation mechanism for deploying the device on the user for delivering medication to the user, wherein the detachable activation mechanism is configured to releasably connect to the housing, wherein the detachable activation mechanism comprises a trigger and wherein the detachable activation mechanism is configured to insert the infusion catheter into the user and to automatically release the detachable activation mechanism from the housing responsive to activation of the trigger.

5. The infusion system of claim 4 wherein the detachable activation mechanism includes a retraction mechanism comprising an introducer needle for introducing the infusion catheter into the user.

6. The infusion system of claim 5 wherein the introducer needle is configured to automatically retract within the needle retraction assembly to shield the needle upon the automatic release of the detachable activation mechanism.

7. The infusion system of claim 4 further comprising a cartridge configured to move with respect to the housing and insert the infusion catheter into the user.

8. The infusion system of claim 4 further comprising a carrier configure to carry the infusion catheter, wherein the needle retraction assembly is mounted on the carrier, wherein the carrier is configured to move in response to movement of the retraction assembly from a first position wherein the infusion catheter is not inserted into the user to a second position wherein the carrier is advanced within the device and the introducer needle is inserted into the user.

9. The infusion system of claim 4 wherein the device is configured to adhere to a skin of the user.

10. The infusion system of claim 7 wherein the detachable activation mechanism includes a retraction mechanism mounted on the cartridge comprising an introducer needle for introducing the infusion catheter into the user.

11. The infusion system of claim 4 wherein the medication is insulin.

12. An infusion system comprising:
a detachable activation mechanism for deploying a device on the user for delivering medication to the user through an infusion catheter and detaching from the device subsequent to deployment of the device, wherein the detachable activation mechanism includes a needle retraction assembly comprising an introducer needle configured to introduce the infusion catheter into the user,
wherein needle retraction assembly is configured to move the introducer needle from (a) a first position wherein the introducer needle is not inserted in the user, to (b) a second position wherein the introducer needle is inserted into the user and the catheter is introduced into the user, and then to (c) a third position wherein the introducer needle is in a retracted position within the needle retraction assembly shielded from the user while the catheter is maintained in the user.

13. The infusion system of claim 12 further comprising:
a carrier configure to carry the infusion catheter, wherein the needle retraction assembly is mounted on the carrier, wherein the carrier is configured to move in response to movement of the retraction assembly from a first position wherein the infusion catheter is not inserted into the user to a second position wherein the carrier is advanced within the device and the introducer needle is inserted into the user.

14. The infusion system of claim 12 wherein the detachable activation mechanism includes a release lever configured to rotate, an insertion spring, an insertion rod configured to move downward and an activation button configured to move in response to pressure from the user causing release lever to rotate freeing the insertion rod to move downward and the compressed insertion spring forces the insertion rod downward, thereby causing the retraction mechanism to move toward the user along with the carrier inserting the infusion catheter or analyte sensor into the user.

15. The infusion system of claim 12 wherein the detachable activation mechanism includes a trigger configured to release the detachable activation mechanism from the device for delivering medication and automatically retract and shield the introducer needle within the needle retraction mechanism.

16. The infusion system of claim 12 wherein the detachable activation mechanism includes a rib and a projection, wherein the rib and projection defining a slot to engage the needle retraction assembly.

17. The infusion system of claim 16 wherein the needle retraction assembly includes a housing having a release lever with a boss extending on distal end of the release lever, wherein the boss is configured to slide along the rib as the needle retraction assembly advances toward the user and enter the slot, thereby causing the release lever to flex outwardly and enabling the introducer needle to retract within the needle retraction assembly.

18. The infusion system of claim 17 wherein the needle retraction assembly further includes a retraction spring configured to retract the introducer needle as the release lever flexes.

19. The infusion system of claim 12 wherein the medication is insulin.

20. The infusion system of claim 13 wherein the carrier is a cartridge assembly configured to move from (1) a telescoping position above device, wherein the first introducer needle is in a retracted position within the device for delivering medication to the user to (2) an advanced position within the device, wherein the introducer needle is in a deployed position inserted into the user along with the catheter.

21. The infusion system of claim 12 wherein the retraction assembly further includes a second introducer needle for introducing an analyte sensor into the user.

22. The infusion system of claim 21 wherein the analyte sensor is a CGM sensor.

23. An infusion system comprising:
a device for delivering medication to a user;
a cartridge assembly including an infusion catheter for infusing the medication into tissue of the user and an analyte sensor for measuring an analyte in the user,
a detachable activation mechanism for deploying the device on the user for delivering medication to the user and for detaching from the device subsequent to deployment of the device,
wherein the detachable activation mechanism comprises a needle retraction assembly mounted on the cartridge assembly, the needle retraction assembly includes a first introducer needle for introducing the infusion catheter into the user and a second introducer needle for introducing the analyte sensor in the user,
wherein the detachable activation mechanism is configured to cause the cartridge assembly to move from (1) a first position wherein the first and second introducer needles are in a retracted position within the device for delivering medication to the user to (2) a second position within the device, wherein the first and second introducer needles are in a deployed position inserted into a subcutaneous tissue of the user's skin along with the infusion catheter and the analyte sensor, respectively.

24. The infusion system of claim 23 wherein the needle retraction mechanism is configured to retract the first and second introducer needles after insertion and upon release from the device for infusing medication to the user.

25. The infusion system of claim 23 wherein the analyte sensor is a CGM sensor.

26. The infusion system of claim 23 wherein the medication is insulin.

27. A detachable activation mechanism for deploying a device on the user for delivering medication to the user and detaching from the device subsequent to deployment of the device, wherein the device for delivering medication including an infusion catheter for infusing the medication into the user, the detachable activation mechanism comprising:
a needle retraction assembly comprising an introducer needle configured to introduce the infusion catheter into the user,
wherein needle retraction assembly is configured to move the introducer needle from (a) a first position wherein the introducer needle along with infusion catheter are not inserted in the user, to (b) a second position wherein the introducer needle is in a deployed position inserted into the user along with the catheter, and then to (c) a third position wherein the introducer needle is in a retracted position within the needle retraction assembly shielded from the user while the catheter is maintained in the user.
